(19) United States
(12) Patent Application Publication
(10) Pub. No.: US 2025/0104838 A1
(43) Pub. Date: Mar. 27, 2025

(54) ACTIVITY SUPPORT DEVICE, ACTIVITY SUPPORT METHOD, AND RECORDING MEDIUM

(71) Applicant: NEC Corporation, Tokyo (JP)

(72) Inventors: Tatsunori NAGASAWA, Tokyo (JP); Masaru Yanai, Tokyo (JP); Seishi Handa, Tokyo (JP); Tomonori Ishida, Tokyo (JP); Makoto Iwasaki, Tokyo (JP); Noriaki Kobayashi, Tokyo (JP); Takashi Kojima, Tokyo (JP); Naoko Kurisaki, Tokyo (JP); Hideki Muramatsu, Tokyo (JP); Motoharu Ohtake, Tokyo (JP); Nanami Sakurai, Tokyo (JP); Yasuyuki Taguchi, Tokyo (JP); Takayuki Takimoto, Tokyo (JP); Noritsugu Yamanaka, Tokyo (JP); Kosei Kobayashi, Tokyo (JP)

(73) Assignee: NEC Corporation, Tokyo (JP)

(21) Appl. No.: 18/897,585

(22) Filed: Sep. 26, 2024

(30) Foreign Application Priority Data
Sep. 27, 2023 (JP) 2023-164177

(51) Int. Cl. G16H 20/30; G16H 40/67

(52) U.S. Cl. CPC G16H 20/30; G16H 40/67

(57) ABSTRACT

In an activity support device, a plan acquisition means acquires a target physical activity amount of a user in a predetermined period and a predictive physical activity amount representing an expected expenditure of a physical activity amount. An expended physical activity amount acquisition means acquires an expended physical activity amount of the user up to a current time in the predetermined period. A total expended physical activity amount acquisition means derives a total expended physical activity amount of the user in the predetermined period, based on the predictive physical activity amount and the expended physical amount. An activity determination means determines a physical activity to the user from among physical activity options by comparing the target physical amount and the total expended physical amount. Accordingly, it is possible for the activity support device to support a user in making a decision regarding the physical activity.

### TECHNICAL FIELD

This disclosure relates to a device which encourages physical activity for a user.

### BACKGROUND ART

In order to maintain good health and reduce inactivity, it is advisable to incorporate moderate physical activity in daily life, such as walking more and taking stairways. Patent Document 1 describes that a predictive achievement amount of physical activity is calculated by a multivariable analysis using a historical step count and activity pattern, and a process is terminated when a target amount of physical activity is exceeded at a time of calculating the predictive achievement physical activity amount. Moreover, Patent Document 2 describes that target intake information of calories is automatically calculated from physical characteristics and attributes of a user, and an advice is given to increase a physical activity time based on the target intake information and energy expenditure information.
[Patent Document 1] Japanese Patent Application Laid-Open under No. JP 2013-239115
[Patent Document 2] Japanese Patent Application Laid-Open under No. JP 2007-094723

### SUMMARY

In the prior art, it is possible to present an exercise menu for a target physical activity amount inferred from attributes or the like, or to display a physical activity amount actually expended; however, it is difficult to expend the target physical activity amount without an error as planned, and the expended physical activity amount does not reach the target physical activity amount, or an expended physical activity amount significantly exceeds the target physical activity amount. In a case where the expended physical activity amount greatly exceeds the target physical activity amount, a new problem arises: a person may find it difficult to sustain the activity due to its excessive demand on a physical strength of that person.

One object of the present disclosure is to support an appropriate physical activity of a user and its continuation by predicting the physical activity amount to be expended in consideration of a future prospect.

According to an example aspect of the present disclosure, there is provided an activity support device including:
at least one memory configured to store instructions; and
at least one processor configured to execute the instructions to:
acquire a target physical activity amount of a user in a predetermined period and a predictive physical activity amount representing an expected expenditure of a physical activity amount;
acquire an expended physical activity amount of the user up to a current time in the predetermined period;
derive a total expended physical activity amount of the user in the predetermined period, based on the predictive physical activity amount and the expended physical amount; and
determine a physical activity to the user from among physical activity options by comparing the target physical amount and the total expended physical amount.

According to another example aspect of the present disclosure, there is provided an activity support method including:
acquiring a target physical activity amount of a user in a predetermined period and a predictive physical activity amount representing an expected expenditure of a physical activity amount;
acquiring an expended physical activity amount of the user up to a current time in the predetermined period;
deriving a total expended physical activity amount of the user in the predetermined period, based on the predictive physical activity amount and the expended physical amount; and
determining a physical activity to the user from among physical activity options by comparing the target physical amount and the total expended physical amount.

According to still another example aspect of the present disclosure, there is provided a non-transitory computer readable recording medium storing a program, the program causing a computer to perform a process including:
acquiring a target physical activity amount of a user in a predetermined period and a predictive physical activity amount representing an expected expenditure of a physical activity amount;
acquiring an expended physical activity amount of the user up to a current time in the predetermined period;
deriving a total expended physical activity amount of the user in the predetermined period, based on the predictive physical activity amount and the expended physical amount; and
determining a physical activity to the user from among physical activity options by comparing the target physical amount and the total expended physical amount.

### Effect

According to the present disclosure, by predicting an expended physical activity amount in consideration of a future prospect, it is possible to support an appropriate physical activity of a user and its continuation.

### BRIEF DESCRIPTION OF THE DRAWINGS

FIG. 1 illustrates a configuration of an activity support system;

FIG. 2A and FIG. 2B illustrate respective hardware configurations of a user terminal and an activity support device;

FIG. 3 illustrates a functional configuration of an activity support device in a first example embodiment.

FIG. 4A and FIG. 4B illustrate examples of an activity plans;

FIG. 5A and FIG. 5B respectively illustrate an activity encouragement screen and an output audio sound;

FIG. 6 is a flowchart of an activity support process in the first example embodiment;

FIG. 7 illustrates a functional configuration of an activity support device in a second example embodiment;

FIG. 8A and FIG. 8B illustrate examples of an activity encouragement screen and an output audio sound in the second example embodiment;

FIG. 9 illustrates a functional configuration of an activity support device in a third example embodiment;

FIG. 10 illustrates an example of a report; and

FIG. 11 illustrates a flowchart of an activity support process in the third example embodiment.

### EXAMPLE EMBODIMENTS

Preferred example embodiments of the present disclosure will be described with reference to the accompanying drawings.

### First Example Embodiment

### [Configuration]

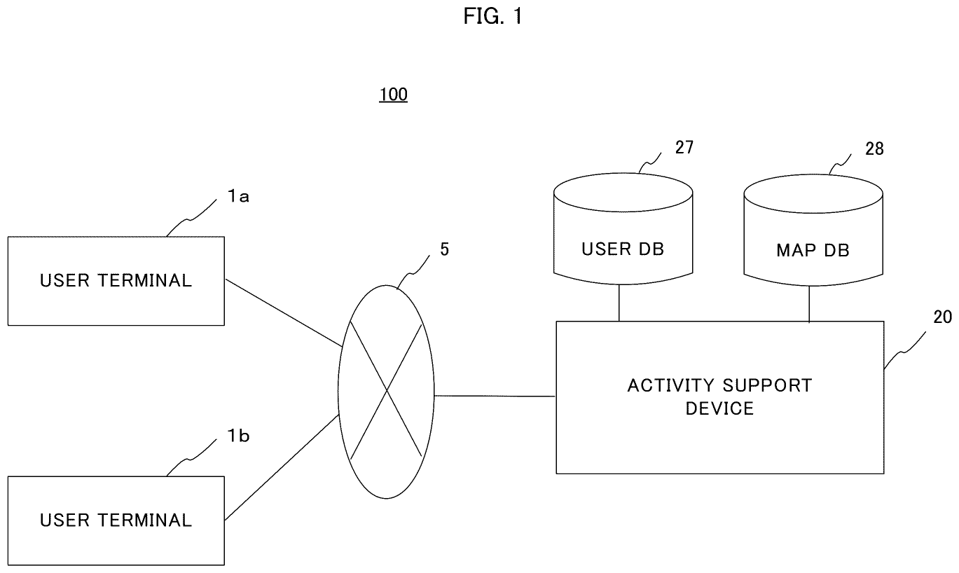

First, a first example embodiment will be described. FIG. 1 illustrates a configuration of an activity support system in which an activity support device of the present disclosure is applied. The activity support system 100 is a system which derives a predictive physical activity amount representing an expected expenditure of a physical activity amount based on an activity plan of a user in a predetermined period, and proposes an appropriate activity to the user as an exercise menu in order to achieve an expenditure of a target physical activity amount.

In the activity support system 100, a user terminal 1a and a user terminal 1b, and the activity support device 20 are mutually communicatively connected via a network 5 such as the Internet. The user terminal 1a and the user terminal 1b are information processing devices used by the same user; in the present example embodiment, the user terminal 1a is a smart phone, and the user terminal 1b is a wearable terminal. Here, it is assumed that the wearable terminal can acquire location information of a current location, a movement distance, an expended physical activity amount, a number of steps, and the like regarding the user. Furthermore, the user terminal 1b automatically transmits the location information of the current location, the movement distance, the expended physical activity amount, the number of steps, and the like regarding the user at a predetermined timing by setting them in advance to the activity support device 20.

The activity support device 20 is an information processing device which processes, stores, and transmits and receives various data. The activity support device 20 proposes physical activities in a daily life as the exercise menu in order to expend the target physical activity amount, and supports the physical activity of the user and its continuation. Examples of activities which expend the physical activity amount in the daily life include walking and using stairways during commuting.

Specifically, the activity support device 20 acquires the activity plan of the user in the predetermined period from the user terminal 1a, and derives the target physical activity amount of the user and the predictive physical activity amount representing an expected expenditure of the physical activity amount. Moreover, the activity support device 20 acquires the expended physical activity amount from the user terminal 1b to the current time in the predetermined period, and derives the total expended physical activity amount of the user in the predetermined period based on the predictive physical activity amount and the expended physical activity amount. Next, the activity support device 20 determines a physical activity recommended to the user at the current time from physical activity options by comparing the target physical activity amount with the total expended physical activity amount, and outputs the determined physical activity to the user terminal 1a.

The activity support device 20 is an information processing device for processing, storing, and transmitting various kinds of information, such as a server device, a personal computer (PC), or a general purpose tablet. The activity support device 20 includes a user database (hereinafter, the database is also referred to as a “DB”) 27 and a map DB 28.

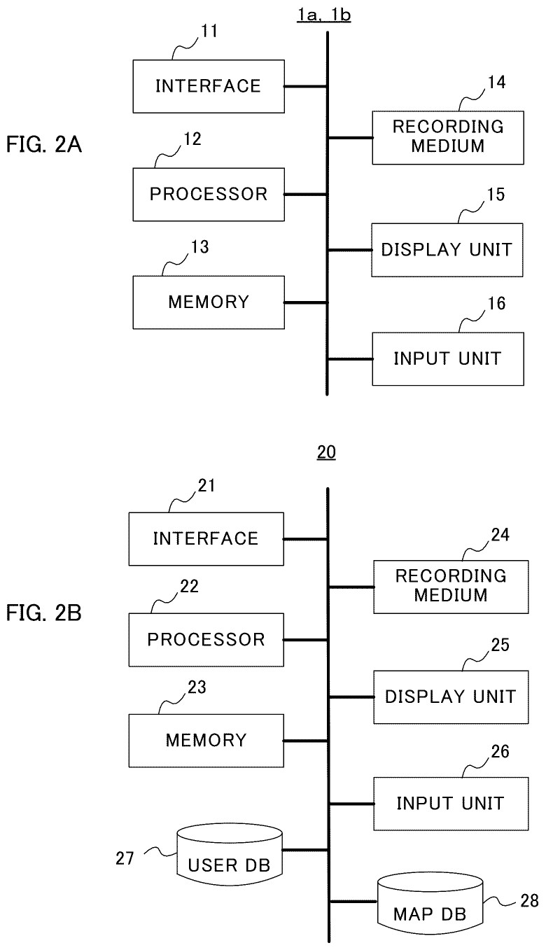

FIG. 2A is a block diagram illustrating a hardware configuration of the user terminal 1a. As illustrated, the user terminal 1a includes an interface (Interface) 11, a processor 12, a memory 13, a recording medium 14, a display unit 15, and an input unit 16. Note that since a hardware configuration of the user terminal 1b is the same as that of the user terminal 1a, the explanations thereof will be omitted for convenience.

The interface 11 exchanges data with the activity support device 20 through the network 5. The interface 11 is used for transmitting various data to the activity support device 20 and receiving various data from the activity support device 20. The processor 12 is a computer such as a CPU (Central Processing Unit) and
controls the entire user terminal 1a by executing programs prepared in advance. The memory 13 is formed by a ROM (Read Only Memory) and a RAM (Random Access Memory). The memory 13 stores programs executed by the processor 12. The memory 13 is also used as a working memory during executions of various processes performed by the processor 12.

The recording medium 14 is a non-volatile and non-transitory recording medium such as a disk-shaped recording medium or a semiconductor memory and is formed to be detachable from the user terminal 1a. The recording medium 14 records various programs executed by the processor 12. The display unit 15 may be an LCD (Liquid Crystal Display) for instance, and displays various information. The input unit 16 may be a touch panel or the like and is used by the user to input various information.

FIG. 2B is a block diagram illustrating a hardware configuration of the activity support device 20. As illustrated, the activity support device 20 includes an interface 21, a processor 22, a memory 23, a recording medium 24, a display unit 25, and an input unit 26. Incidentally, these components and the user DB 27 and the map DB 28 are connected to each other via a bus.

The interface 21 exchanges data with the user terminal 1a and the user terminal 1b through the network 5. The processor 22 is a computer such as a CPU, and controls the entire activity support device 20 by executing programs prepared in advance. The memory 23 is formed by a ROM, a RAM or the like. The memory 23 stores the programs executed by the processor 22. The memory 23 is also used as a working memory during executions of various processes by processor 22.

The recording medium 24 is a non-volatile and non-transitory recording medium such as a disk-shaped recording medium, a semiconductor memory, or the like, and is formed to be detachable to the activity support device 20. The recording medium 24 records various programs executed by the processor 22.

The display unit 25 may be an LCD (Liquid Crystal Display), for instance, and displays each predetermined image. The input unit 26 may be a keyboard, a mouse, a touch panel, or the like, for instance, which is used by an operator who manages the activity support device 20.

The user DB 27 stores information concerning each user such as a password and attributes of the user, which is associated with a user ID identifying the user. Here, the attributes of the user such as gender, age, height, and weight are used as information which is used to derive the target physical activity amount or the expended physical activity amount.

The map DB 28 stores map information for displaying a map on the user terminal 1a. The map information includes road information, facility information, and the like in an area where the user is active. The road information includes, for instance, intersection node data corresponding to an intersection and road link data corresponding to a road with the intersection as an end point. In this case, the intersection node data includes a latitude, a longitude, an altitude, and the like of the intersection. The road link data may include a road width, a slope, a road condition, and like. The facility information may include the location information (latitude and longitude) of the facility, and may include the number of floors and a presence or absence of a transport means (stairways, an elevator, an escalator, or the like) between floors in a case where the facility is a building, or the like. In addition, the map information stores information concerning the transport means used by the user, for instance, information concerning a route of a train or a bus.

Note that the activity support device 20 includes the user DB 27 and the map DB 28 for convenience of explanation; however, the present disclosure is not limited thereto, and as long as required data can be acquired, a type of DB and a data structure of each DB are optional.

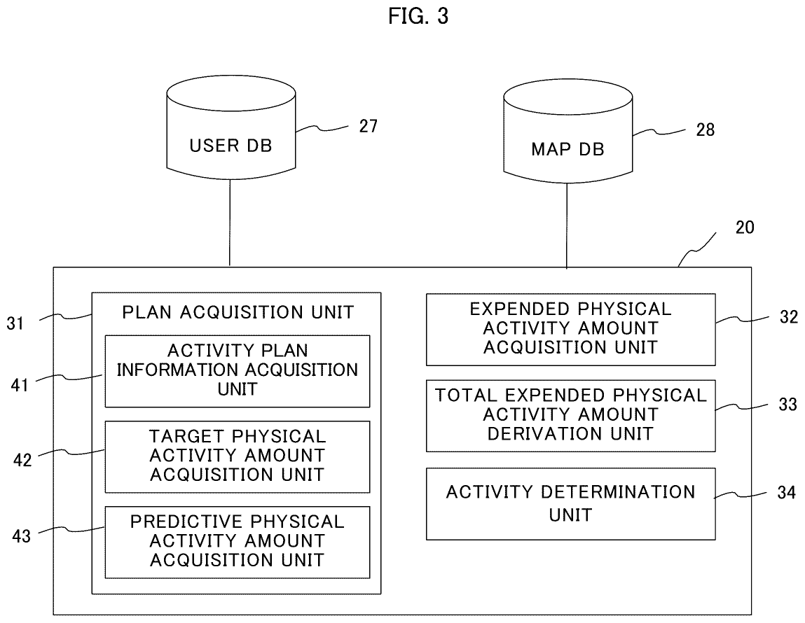

FIG. 3 is a block diagram illustrating a functional configuration of the activity support device 20. The activity support device 20 functionally includes a plan acquisition unit 31, an expended physical activity amount acquisition unit 32, a total expended physical activity amount derivation unit 33, and an activity determination unit 34, and is realized by the processor 12 executing corresponding programs.

The plan acquisition unit 31 includes an activity plan information acquisition unit 41, a target physical activity amount acquisition unit 42, and a predictive physical activity amount acquisition unit 43. Note that the predetermined time period corresponds to a time period which can be arbitrarily set by the user using the user terminal 1a, and indicates “1 day” in the present example embodiment.

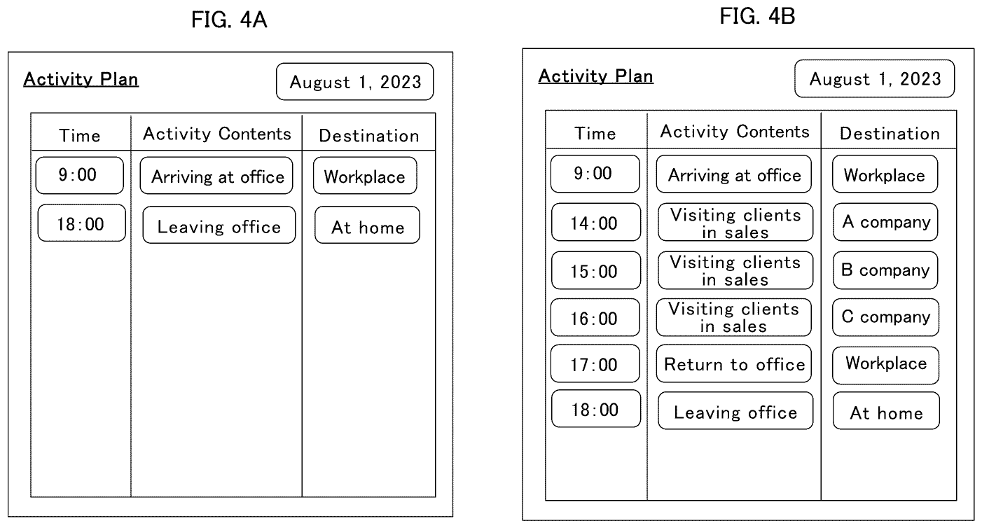

The activity plan information acquisition unit 41 acquires the activity plan information concerning the user ID, the password, and the activity plan for the predetermined period from the user terminal 1a used by the user. FIG. 4A and FIG. 4B illustrate examples of the activity plans. Since the activity support device 20 is intended to expend the target physical activity amount of the user in an activity in daily life rather than a special physical activity, the activity plan is formed by, for instance, a time from leaving home to return to home, activity contents leading to an expenditure of the physical activity amount, and the destination. In response to inputs of the time, the activity contents, and the destination as the activity plan by a predetermined operation of the user using the user terminal 1a, the user terminal 1a transmits the activity plan information to the activity support device 20. Specifically, in the case of an in-house work at a place of work, the user inputs “Arriving at office” and “Leaving office” in the activity contents, along with respective times and destinations. On the other hand, in a case of visiting clients, as illustrated in FIG. 4B, the user inputs not only “Arriving at office” and “Leaving office” but also “Visiting clients in sales” and “Return to office” in the activity contents, and inputs the respective times and destinations together.

Incidentally, addresses and the nearest stations respective to the place of work, home, client companies, which are to be the destinations may be stored in advance by associating the user ID with them in the user DB 27 or a predetermined DB; alternately, an address and the nearest station of each destination may be further input by the user as the activity plan. In addition, the activity plan may be reviewed and modified by the user at any timing during the predetermined period.

The target physical activity amount acquisition unit 42 acquires the target physical activity amount for the predetermined period based on the attributes of the user. The acquired target physical activity amount may be stored in association with the user ID in the user DB 27 or the predetermined DB. The target physical activity amount corresponds to a physical activity amount as a reference for maintaining good health. In the present example embodiment, the target physical activity amount for the predetermined period is set in advance for each gender or age, and the target physical activity amount acquisition unit 42 is able to acquire the target physical activity amount based on the attributes of the user from the memory 23 or the predetermined DB.

Note that in the present example embodiment, as an example, the physical activity amount is assumed to be calculated based on an intensity of the physical activity and time spent performing the physical activity. In this calculation, by changing coefficients of a formula for calculating the physical activity amount based on the agenda, the age, the weight, and the like, it is possible to reflect the attributes of the user to the physical activity amount. Here, the intensity of the physical activity is a degree of tightness which a body feels when the body is moved, and the intensity of the physical activity for each activity may be defined in advance, such as 1 MET at rest, 3 METs for ordinary walking on a level ground, 5 METs for faster walking on the level ground, and the like.

The predictive physical activity amount acquisition unit 43 derives and acquires the predictive physical activity amount which represents the expected expenditure of the physical activity amount from the current time until the end of the predetermined period, based on the activity plan of the predetermined period. Specifically, the predictive physical activity amount acquisition unit 43 specifies a transport means for the user in the predetermined period and a distance with respect to a route to move, based on the activity plan by referring to the map DB 28, and derives the predictive physical activity amount from the current time to an end of the predetermined period. For instance, in a case of the activity plan as illustrated in FIG. 4A, the predictive physical activity amount acquisition unit 43 specifies a distance of a route to be moved by walking in a round trip between home and the place of work, and derives the predictive physical activity amount in reference to a walking time acquired based on the intensity of the physical activity by the ordinary walking and the distance. In addition, in a case of the activity plan as illustrated in FIG. 4B, the predictive physical activity amount acquisition unit 43 specifies a distance for a route among Company A, Company B and Company C to be traveled on foot in addition to the round trip between home and the place of work, and derives the predictive physical activity amount in reference to a walking time based on the intensity of the physical activity by ordinary walking and distance.

Note that since the predictive physical activity amount changes from time to time, the predictive physical activity amount acquisition unit 43 may acquire the predictive physical activity amount at any timing, such as every minute or every 10 minutes, or may acquire them at a time of an activity determination to be described later.

The expended physical activity amount acquisition unit 32 acquires the expended physical activity amount which represents an expenditure of the physical activity amount by the user up to the current time in the predetermined period. Specifically, the expended physical activity amount acquisition unit 32 acquires the expended physical activity amount from the user terminal 1b which is the wearable terminal. At this time, the expended physical activity amount acquisition unit 32 may acquire the expended physical activity amount directly from the wearable terminal, or may acquire the expended physical activity amount from the location information of the user, the movement distance, the number of steps, vital information, and the like. Note that since the expended physical activity amount changes from time to time, the expended physical activity amount acquisition unit 32 may acquire the expended physical activity amount from the user terminal 1b at any timing such as 1 minute unit, 10 minutes unit, or the like, and may acquire the expended physical activity amount at the time of the activity determination to be described later.

The total expended physical activity amount derivation unit 33 predicts and derives the total expended physical activity amount which is expended by the user in the predetermined period based on the predictive physical activity amount and the expended physical activity amount. Specifically, the total expended physical activity amount derivation unit 33 derives the total expended physical activity amount by adding the expended physical activity amount from a start of the predetermined period to the current time and the predictive physical activity amount from the current time to the end of the predetermined period. Note that since the total expended physical activity amount changes from time to time, the total expended physical activity amount derivation unit 33 may acquire the total expended physical activity amount at any timing such as 1 minute unit, 10 minutes unit, or the like, and may acquire the expended physical activity amount at the time of the activity determination to be described later.

The activity determination unit 34 compares the target physical activity amount with the total expended physical activity amount at a predetermined timing during the predetermined period, and newly determines and updates the physical activity recommended to the user from among the physical activity options so that the total expended physical activity amount matches the target physical activity amount. The activity determination unit 34 generates activity encouragement information for encouraging the user to perform the determined physical activity in consideration of the current location of the user and the activity plan, and transmits the activity encouragement to the user terminal 1a. Accordingly, it is possible for the user terminal 1a to display an activity encouragement screen which encourages the determined physical activity based on the activity encouragement information, and to output an audio sound. In other words, the activity support device 20 may propose the determined physical activity to the user as the exercise menu.

Here, the physical activity options represent options of activities to expend the physical activity amount. In the present example embodiment, the timing at which the activity determination unit 34 determines the physical activity is when the user moves from the current location to the destination, but the present disclosure is not limited thereto, and the user may designate a time arbitrarily. Specifically, the physical activity options include various physical activities with different physical activity amount to expend such as ordinary walking at the shortest distance to the destination, taking a long away to the destination and walking faster, and the like, and can be arbitrarily set.

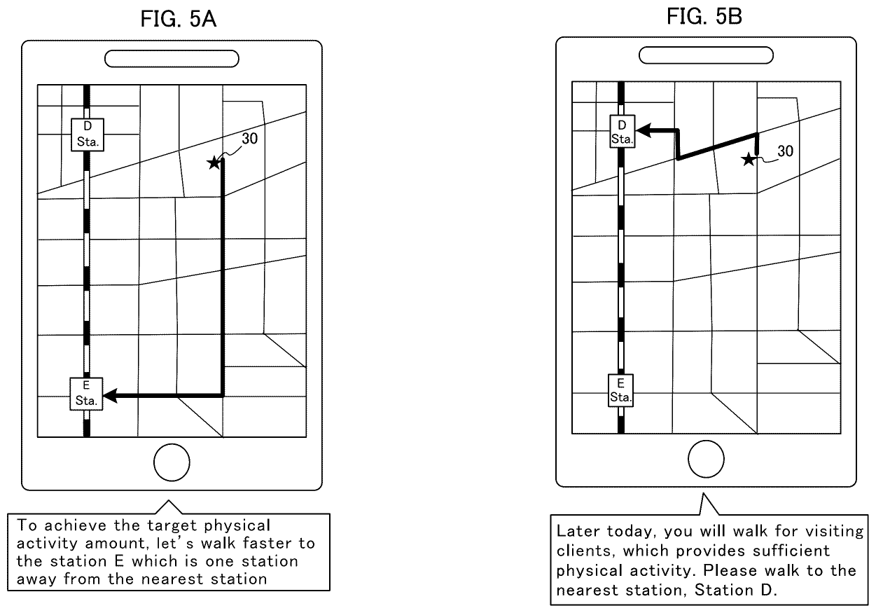

FIG. 5A and FIG. 5B are examples of the activity encouragement screens and the output audio sounds. Specifically, in a case where the activity contents as depicted in FIG. 4A, since the activity is the in-house work and the predictive physical activity amount per day is low, the user needs to expend a lot of physical activity amount when communing between the home and the place of work in order to expend the target physical activity amount. Therefore, when the user commutes from the home to the place of work at the start of the predetermined period, the activity determination unit 34 determines an option of “taking a long way to the destination and walking faster” in which the activity amount is expended more is determined to recommend to the user from among the physical activity options. The activity determination unit 34 generates the activity encouragement information for encouraging the user to perform the determined physical activity in consideration of the location information of the current location of the user and the activity plan, and transmits the activity encouragement information to the user terminal 1a. Based on the received activity encouragement information, the user terminal 1a displays the activity encouragement screen indicating a route from home 30 on a map to Station E, which is one station away from the nearest station due to a detour, as depicted in FIG. 5A. At this time, the user terminal 1a outputs an audio sound such as “To achieve the target physical activity amount, let's walk faster to Station E which is one station away from the nearest station.”. Contents of this audio sound may be displayed as a text message on the screen.

On the other hand, in a case of the activity contents as illustrated in FIG. 4B, in addition to the in-house work, there is a lot of the predictive physical activity amount on that day due to visiting clients by walking around. Thus, in a case where the user spends a lot of the physical activity amount when commuting between home and the place of work, the physical activity amount expended may significantly exceed the target physical activity amount. For this reason, in a case where the user who is at the start of the predetermined period moves to the place of work from home, the activity determination unit 34 determines “Ordinary walking at the shortest distance to the destination” as the physical activity recommended to the user from among the physical activity options in which the physical activity amount to be expended is small. Next, the activity determination unit 34 generates the activity encouragement information for encouraging the determined physical activity by considering the location information of the current location of the user and the activity plan, and transmits the information to the user terminal 1a. Based on the received activity encouragement information, as illustrated in FIG. 5B, the user terminal 1a displays the activity encouragement screen indicating the route from home 30 on a map to Station D which is the nearest station. At this time, the user terminal 1a outputs an audio sound such as “Later today, you will walk for visiting clients, which provides sufficient physical activity. Please walk to the nearest station, station D.”. Contents of this audio sound may be displayed as a text message on the screen.

As described above, since an aim is to correspond the physical activity amount actually expended by the user to the target physical activity amount in the predetermined period without excess or deficiency, in a case where the total expended physical activity amount is sufficient with respect to the target physical activity amount, the activity support device 20 may determine an effortless physical activity with less physical activity amount as an activity to recommend, and propose the effortless physical activity to the user.

### (Activity Support Process)

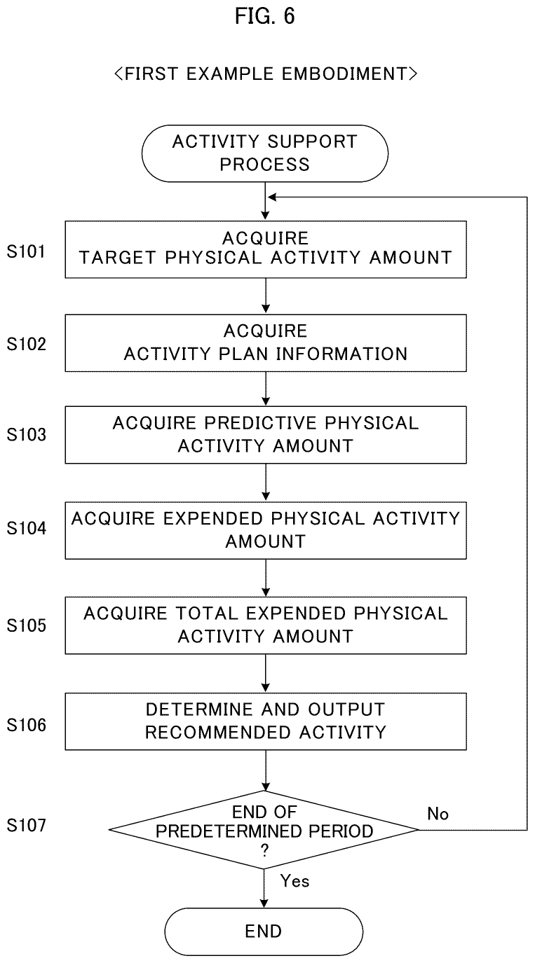

Next, an activity support process by the activity support device 20 will be described. FIG. 6 is a flowchart of the activity support process performed by the activity support device 20. This process is realized by the processor 22 illustrated in FIG. 2B, which executes a corresponding program prepared in advance.

First, the user logs in to a predetermined website or the like, or starts a predetermined application by operating the user terminal 1a and inputting the user ID and the password. Thus, the activity support device 20 acquires the user ID and the password. Then, the activity support device 20 refers to the user DB 27 based on the acquired user ID, and acquires the target physical activity amount for the predetermined period based on the attributes of the user (step S101).

Furthermore, the user inputs the activity plan in the predetermined period from the predetermined website or the predetermined application. Thus, the activity support device 20 acquires the activity plan information of the user in the predetermined period (step S102). Based on the activity plan included in the acquired activity plan information, the activity support device 20 derives and acquires the predictive physical activity amount from the current time to the end of the predetermined period (step S103). Moreover, the activity support device 20 acquires the expended physical activity amount expended by the user in the predetermined period by the current time (step S104). Then, the activity support device 20 derives and acquires the total expended physical activity amount to be expended by the user in the predetermined period based on the predictive physical activity amount and the expended physical activity amount of the current time (step S105).

The activity support device 20 compares the target physical activity amount with the total expended physical activity amount at a timing when the user moves from the current location to the destination, and determines the physical activity recommended to the user from among the physical activity options such that the total expended physical activity amount corresponds to the target physical activity amount. Then, the activity support device 20 generates the activity encouragement information for encouraging the determined physical activity, and transmits the information to the user terminal 1a (step S106). Thus, the user terminal 1a displays the activity encouragement screen which encourages the user to take the physical activity based on the activity encouragement information, and outputs the audio sound. In other words, the activity support device 20 proposes the determined physical activity to the user as the exercise menu.

Next, the activity support device 20 determines whether or not the predetermined period ends (step S107). When it is determined that the predetermined period has not ended (step S107; No), the activity support device 20 goes back to a process of step S101. On the other hand, when it is determined that the predetermined period ends (step S107; Yes), the activity support device 20 terminates the activity support process.

Note that in the present example embodiment, the user uses the smart phone as the user terminal 1a and the wearable terminal as the user terminal 1b; however, the present disclosure is not limited thereto, and only the smart phone which is the user terminal 1a may be used by the user. In this case, since the expended physical activity amount cannot be acquired from the wearable terminal which is the user terminal 1b, the expended physical activity amount acquisition unit 32 acquires the location information of the current location, the movement distance, the number of steps, and the like of the user at any timing from the smart phone which is the user terminal 1a, and calculates the expended physical activity amount.

Moreover, in the present example embodiment, the activity support device 20 acquires the activity plan information by the user inputting the activity plan for the predetermined period from the predetermined website or the predetermined application. However, the present disclosure is not limited thereto, and the activity support device 20 may acquire the activity plan information from a predetermined schedule application or a predetermined DB.

Moreover, in the present example embodiment, the target physical activity amount acquisition unit 42 acquires the target physical activity amount based on the attributes of the user; however, the present disclosure is not limited thereto, it may be obtained target physical activity amount arbitrarily set by the user. In this time, the target physical activity amount acquisition unit 42 may display the target physical activity amount based on the attributes of the user on the user terminal 1a as a reference.

Moreover, in the present example embodiment, the physical activity amount is calculated based on the intensity of the physical activity and the time of performing the physical activity amount; however, the present disclosure is not limited thereto, the physical activity amount may be calculated by an arbitrary calculation formula, and may be calories to be expended.

As described above, the activity support device 20 can predict the future expended physical activity amount in the predetermined period based on the activity plan. Therefore, the activity support device 20 can derive the total expended physical activity amount with a more accuracy by adding a physical activity amount expended up to that time and an expected expenditure of the physical activity amount. Accordingly, it is possible for the activity support device 20 to suggest the physical activity with the most appropriate amount at that point in time as the exercise menu. By performing the proposed exercise menu, it is possible for the user to expend an adequate physical activity amount as needed by daily activities without actually expended physical activity amount falling below or significantly exceeding the target physical activity amount.

As described above, it is possible for the activity support device 20 to incorporate healthcare into daily life by proposing the recommended physical activity to the user as the exercise menu at the appropriate timing in consideration of the activity plan, thereby making it possible for the user to make the physical activity a habit without strain.

### Second Example Embodiment

Next, a second example embodiment will be described. The activity support device 20 according to the first example embodiment refers to the map DB 28, and determines the distance for the route of the user moving by the transport means in a predetermined period based on the activity plan to derive the predictive physical activity amount. However, an activity support device 20x of the second example embodiment refers to the map DB 28, and derives the predictive physical activity amount in consideration of not only the distance of the route of the user but also environmental information such as a variation of a load on a road surface condition in the route. Note that in the present example embodiment, for convenience of explanation, only portions different from those in the first example embodiment will be described.

A hardware configuration of the activity support device 20x of the present example embodiment is the same as that of the activity support device 20 of the first example embodiment. Similar to the first example embodiment, the map DB 28 stores the map information including the road information, the facility information, and the like in an area where the user is active. In addition, in the second example embodiment, the map DB 28 includes the environmental information concerning roads and facilities. Specifically, the road information includes, as the environmental information, the road surface condition, the variation in the load on the road surface condition, and the like. The road surface condition includes, for instance, whether the road is paved or rough, whether the road has a slope, a footbridge, a crosswalk, stairways, and the like. The load on the road surface condition is indicated by, for instance, a numerical value set based on a difficulty when moving with normal walking in which a large numerical value is set for the slope with a large gradient and a small numerical value is set for a flat road surface being paved. Compared to a flat road surface, the stairways are set with a large number, and an escalator and an elevator are set with a small value. Also, the facility information also indicates, as the environmental information, a presence or absence of the stairways, the escalator, the elevator, and the like between respective floors in the facility.

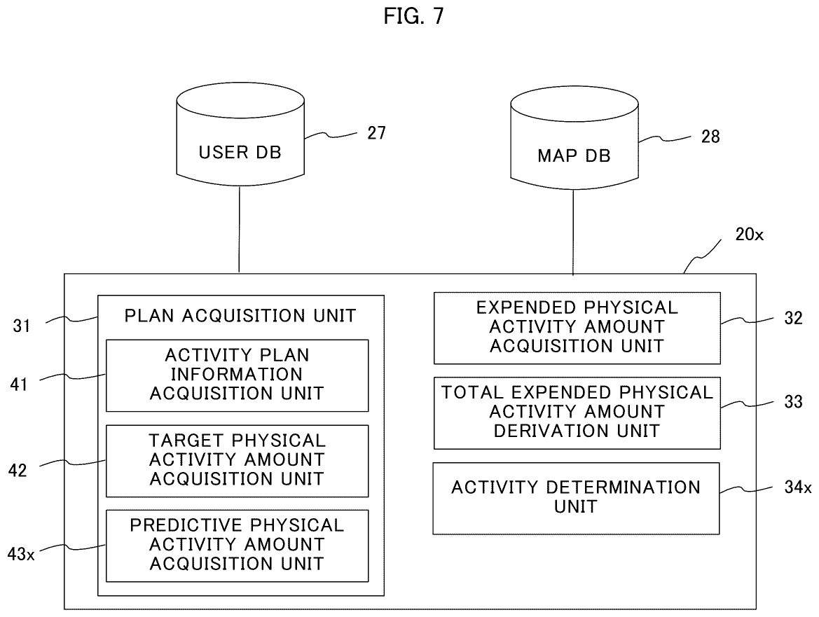

FIG. 7 is a block diagram illustrating a functional configuration of the activity support device 20x. The activity support device 20x functionally includes a plan acquisition unit 31, an expended physical activity amount acquisition unit 32, a total expended physical activity amount derivation unit 33, and an activity determination unit 34x, and is realized by the processor 22 executing corresponding programs.

The plan acquisition unit 31 includes an activity plan information acquisition unit 41, a target physical activity amount acquisition unit 42, and a predictive physical activity amount acquisition unit 43x. Note that the predetermined time period is a time period which can be arbitrarily set by the user using the user terminal 1a, and is “1 day” in the present example embodiment.

The predictive physical activity amount acquisition unit 43x derives and acquires the predictive physical activity amount representing the expected expenditure of the physical activity amount from the current time to the end of the predetermined period, based on the activity plan for the predetermined period. Specifically, the predictive physical activity amount acquisition unit 43 refers to the map DB 28, specifies the transport means for the user during the predetermined period from the activity plan, the distance of the route to move, and the variation in the load on the road surface condition in the route to move, and derives the predictive physical activity amount from the current time to the end of the predetermined period. For instance, in a case of the activity plan as illustrated in FIG. 4A, the predictive physical activity amount acquisition part 43 specifies the distance of the route to move by walking in the round trip between home and the place of work, and derives the predictive physical activity amount by multiplying the intensity of the physical activity of the ordinary walking by a time taken for the distance in the ordinary walking. At this time, the predictive physical activity amount acquisition unit 43x reflects a numerical value indicating the load on the road surface condition in the route to move as a coefficient. Thus, the predictive physical activity amount acquisition unit 43x can derive the predictive physical activity amount of the route in which the load on the road surface condition is great to be a large value, and can derive the predictive physical activity amount of the route in which the load on the road surface condition is small to be a small value.

Thus, in the present example embodiment, since the environmental information is considered such as variation in the load on the road surface condition in the route to move, it is possible to set various activities each which expends a different physical activity amount, as the physical activity options, such as a activity selecting the footbridge rather than a crosswalk and selecting the elevator rather than the stairway when there are a plurality of transport means which differ in the load on the road surface condition. Therefore, in the present example embodiment, the activity determination unit 34x refers to the map DB 28 based on the location information of the current location of the user, and also determines the physical activity to be recommended to the user from the physical activity options when the user approaches a place where a plurality of transport means with different road surface conditions are available. Next, the activity determination unit 34x generates the activity encouragement information for encouraging the physical activity determined in consideration of the activity plan of the user, and sends the activity encouragement information to the user terminal 1a. Thus, the user terminal 1a can display the activity encouragement screen which encourages the user to perform the determined physical activity, based on the activity encouragement information, and output the audio sound. In other words, it is possible for the activity support device 20x to propose the determined physical activity to the user as the exercise menu.

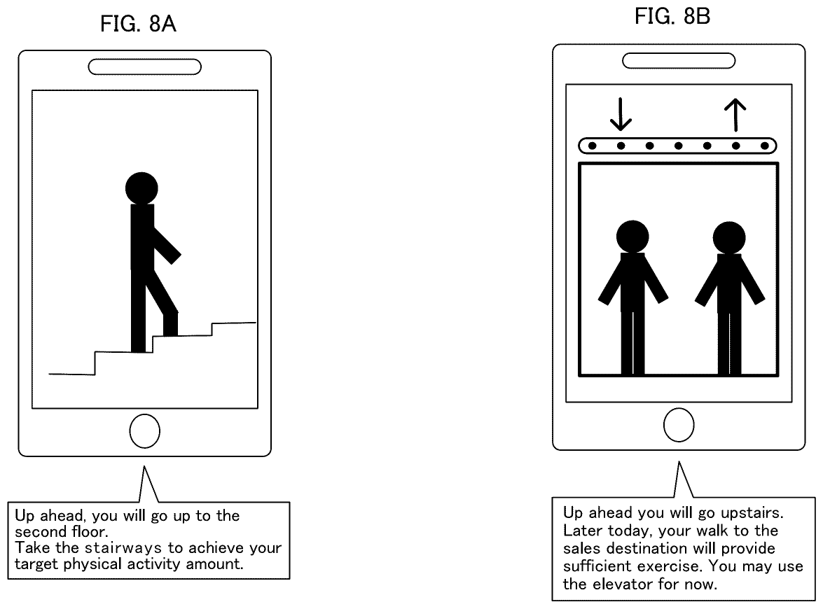

FIG. 8A and FIG. 8B illustrates examples of the activity encouragement screen and the output audio sound in the second example embodiment. Specifically, in a case of the activity contents as illustrated in FIG. 4A, the predictive physical activity amount for one day is small due to the in-house work and thus, the user needs to expend the target physical activity amount more when the user moves. Therefore, in a case where the user approaches a place to use one of the stairways and the elevator, the activity determination unit 34x determines, as the physical activity to be recommended to the user, an activity “use the stairways instead of the elevator” which expends the physical activity amount more from among the physical activity options based on the location information of the current location of the user. The activity determination unit 34x then generates the activity encouragement information for encouraging the determined physical activity in consideration of the activity plan of the user, and sends the activity encouragement information to the user terminal 1a. Based on the received activity encouragement information, the user terminal 1a displays the activity encouragement screen representing the use of the stairways as illustrated in FIG. 8A, and outputs an audio sound such as “Up ahead, you will go up to the second floor. Take the stairways to achieve your target physical activity amount.”. Contents of this audio sound may be displayed as a text message on the screen.

On the other hand, in a case of the activity contents as illustrated in FIG. 4B, in addition to the in-house work, there is a lot of the predictive physical activity amount on that day due to visiting clients by walking around. Thus, the physical activity amount which the user expends may be great and significantly exceed the target physical activity. Therefore, the activity determination unit 34x determines, as the physical activity recommended to the user, an activity “use the elevator instead of the stairways” which expends less physical activity amount from among the physical activity options when the user approaches a place where one of the stairways and the elevator can be used based on the location information of the current location of the user. Next, the activity determination unit 34x generates the activity encouragement information for encouraging the determined physical activity in consideration of the activity plan of the user, and transmits the activity encouragement information to the user terminal 1a. Based on the received activity encouragement information, the user terminal 1a displays the activity encouragement screen representing the use of the elevator as illustrated in FIG. 8B, and outputs an audio sound such as “Up ahead you will go upstairs. Later today, your walk to the sales destination will provide sufficient exercise. You may use the elevator for now.”. Contents of this audio sound may be displayed as a text message on the screen.

Since the activity support process by the activity support device 20x is the same as the activity support process by the activity support device 20 in the first example embodiment, explanations thereof will be omitted for convenience.

As described above, according to the activity support device 20x, based on the activity plan of the user acquired in advance, it is possible to predict an expenditure of the physical activity amount expected over the predetermined period in consideration of not only the distance of the route to the destination but also the environmental information such as the variation in the load on the road surface conditions on the route. Moreover, it is possible for the activity support device 20x to propose various activities in daily life to the user as the exercise menu based on the environmental information.

### Third Example Embodiment

Next, a third example embodiment will be described. The activity support device 20y according to the third example embodiment notifies the user of a request of an evaluation of a quality of a physical activity level when the predetermined period ends. Here, the quality of the physical activity level indicates a physical tightness felt for the physical activity proposed as the exercise menu. Next, the activity support device 20y derives a change proposal of the target physical activity amount according to a result of a quality evaluation by the user. In the present example embodiment, for convenience of explanation, only portions different from those in the first example embodiment will be described.

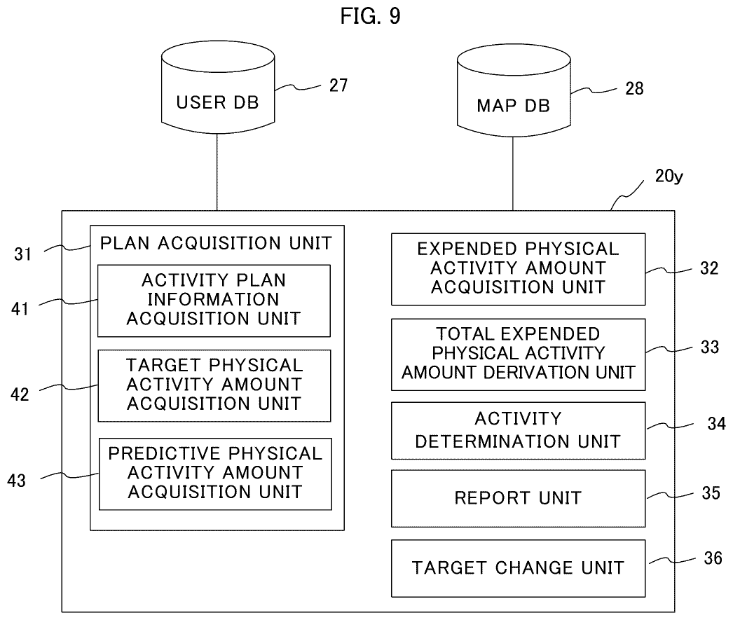

FIG. 9 is a block diagram illustrating a functional configuration of the activity support device 20y. The activity support device 20y functionally includes the plan acquisition unit 31, the expended physical activity amount acquisition unit 32, the total expended physical activity amount derivation unit 33, the activity determination unit 34, a report unit 35, and a target change unit 36, and is realized by the processor 22 executing corresponding programs.

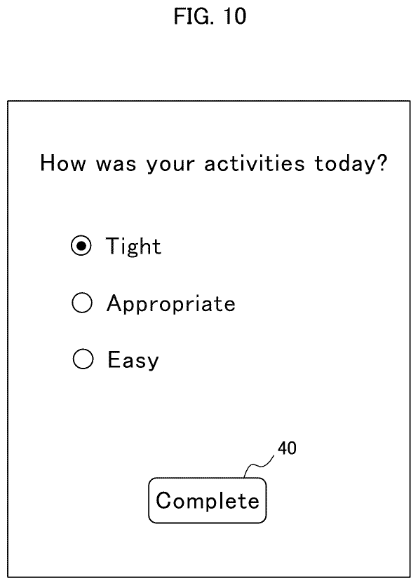

FIG. 10 is an example of a report screen. When the predetermined period ends, the report unit 35 transmits the report screen as illustrated in FIG. 10 to the user terminal 1a in order to request the user to evaluate the quality of the physical activity level. When the user terminal 1a displays report items on the report screen, the user selects an appropriate one from three stages; “Tight”, “Appropriate”, and “Easy” according to the predetermined operations, and presses the completion button 40. Thus, the user terminal 1a transmits a selection result by the user to the activity support device 20y as a result of the quality evaluation for the physical activity level. Note that the report screen illustrated in FIG. 10 is an example, and the form of requesting the quality evaluation for the physical activity level to the user can be arbitrarily set.

The target change unit 36 acquires the result of the quality evaluation by the user from the user terminal 1a, and derives a change proposal of the target physical activity amount of the user which is acquired by the target physical activity amount acquisition unit 42 in accordance with the result of the quality evaluation. For instance, when the result of the quality evaluation by the user indicates “Easy”, the target change unit 36 derives a change proposal for increasing the target physical activity amount for a following predetermined period. On the other hand, when the result of the quality evaluation by the user is “Tight”, the target change unit 36 derives a change proposal for reducing the target physical activity amount for the following predetermined period. When the change proposal of the target physical activity amount is derived, the activity support device 20y changes the target physical activity amount stored in the user DB 27 or the predetermined DB in association with the user ID based on the change proposal, and proposes the changed target physical activity amount to the user by displaying a screen or outputting the audio sound in the following predetermined period. Thus, the activity support device 20y can propose more appropriate activities as the exercise menu according to the result of the quality evaluation by the user.

Note that, in the present example embodiment, the target change unit 36 changes the target physical activity amount according to the result of the quality evaluation by the user; however, the present disclosure is not limited thereto, and the target change unit 36 may change the intensity for each physical activity proposed as the exercise menu. For instance, in a case where the result of the quality evaluation by the user is “Tight”, by lowering the intensity of the physical activity to be proposed and increasing a time length for the physical activity to be proposed, it is possible to propose an appropriate physical activity for the user without changing the target physical activity amount.

### (Activity Support Process)

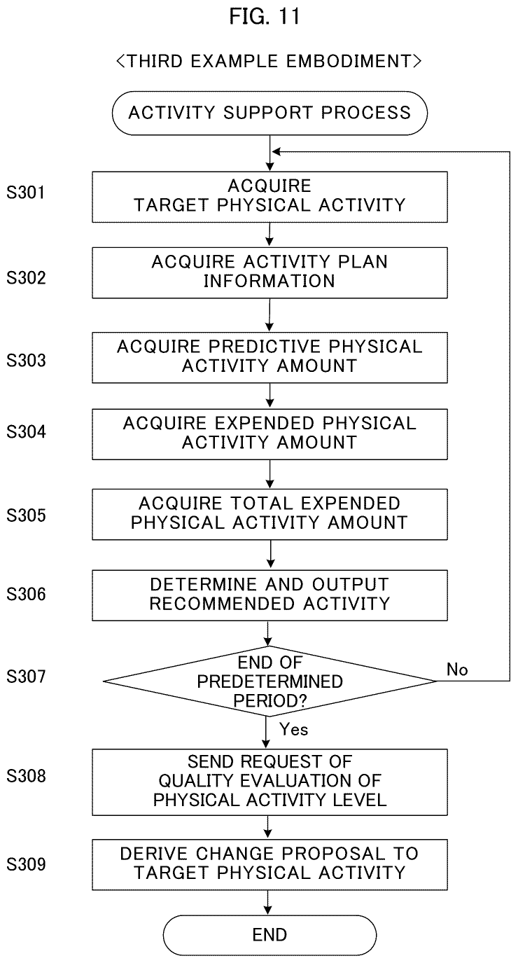

Next, the activity support process by the activity support device 20y will be described. FIG. 11 is a flowchart for explaining the activity support process by the activity support device 20y. This activity support process is realized by the processor 22 illustrated in FIG. 2B, which executes a corresponding program prepared in advance.

First, the user logs in to a predetermined website or the like or starts a predetermined application by operating the user terminal 1a and inputting the user ID and the password. Thus, the activity support device 20y acquires the user ID and the password. Next, the activity support device 20y refers to the user DB 27 based on the acquired user ID, and acquires the target physical activity amount for a predetermined period based on the attributes of the user (step S301).

Furthermore, the user inputs the activity plan in the predetermined period from the predetermined website or the predetermined application. Thus, the activity support device 20y acquires the activity plan information of the user in the predetermined period (step S302). Based on the activity plan indicated in the acquired activity plan information, the activity support device 20y derives and acquires the predictive physical activity amount from the current time until the end of the predetermined period (step S303). Moreover, the activity support device 20y acquires the expended physical activity amount representing the physical activity amount expended by the user up to the current time in the predetermined period (step S304). Next, the activity support device 20y derives and acquires the total expended physical activity amount expended by the user in the predetermined period based on the predictive physical activity amount and the expended physical activity amount (step S305).

The activity support device 20y compares the target physical activity amount with the total expended physical activity amount, at a timing when the user moves from the current location to the destination, and determines the physical activity recommended to the user from among the physical activity options so that the total expended physical activity amount matches the target physical activity amount, Next, the activity support device 20y generates the activity encouragement information for encouraging the determined physical activity, and transmits the activity encouragement information to the user terminal 1a (step S306). Thus, the user terminal 1a displays the activity encouragement screen which encourages the user to perform the determined physical activity based on the activity encouragement information, and outputs the audio sound.

Next, the activity support device 20y determines whether or not the predetermined period ends (step S307). When it is determined that the predetermined period has not ended (step S307; No), the activity support device 20y goes back to the process of the step S301. On the other hand, when it is determined that the predetermined time period ends (step S307; Yes), the activity support device 20y transmits the report screen for requesting to evaluate the quality of the physical activity level to the user terminal 1a (step S308). When the result of the quality evaluation is acquired from the user terminal 1a in accordance with operation of the user, the activity support device 20y derives the change proposal of the target physical activity amount according to the result of the quality evaluation (step S309), and the activity support process is terminated.

Note that the activity support process of the present example embodiment is not limited to the first example embodiment, and can be applied to the second example embodiment.

### [First Modification]

In the first example embodiment described above, the user inputs the destination as the activity plan; however, the present disclosure is not limited thereto, and the user may input the route to the destination. The user may freely change the route proposed by the activity support device 20 on the activity encouragement screen as illustrated in FIG. 5, for instance.

In this case, the predictive physical activity amount acquisition unit 43 derives the predictive physical activity amount in consideration of the route input or changed by the user. According to this, for instance, it is possible to reflect a desire of the user to the activity plan such as “because feel fine in the morning, it is preferable to walk to Station E which is one station away from the nearest station when going to the office”, “because not feel well in the morning, it is preferable to walk to the shortest distance when going to the office”, or the like. Therefore, it is possible for the activity support device 20 to propose a necessary and sufficient activity to the user as the exercise menu at an appropriate timing in consideration of the desire of the user.

The activity support device 20 may display an avatar on the route proposed by the activity support device 20 on the activity encouragement screen as illustrated in FIG. 5. By accompanying the avatar on the route to the destination, it is possible to visualize an ideal physical activity and it is possible for the user to prompt to select a route with a high activity effect.

### [Second Modification]

The activity support device 20 may send an alert to the user terminal 1a to prompt the user to modify the activity plan when the location information of the current location of the user differs from the location information of the user indicated by the activity plan at that time. When the user terminal 1a receives the alert, the user terminal 1a displays a message “Has the activity plan changed? Modify the activity plan.” on the screen.

When the activity plan is modified by the user, the predictive physical activity amount acquisition unit 43 derives the predictive physical activity amount based on the modified activity plan. Moreover, even in a case where the activity plan is not modified by the user, for instance, when the location information of the current location of the user does not indicate Company A of a sales destination but the place of work, the predictive physical activity amount acquisition unit 43 derives the predictive physical activity amount as the in-house work in a time zone for vising Company A specified in the activity plan.

As described above, according to this modification, even when a state is different from an initial activity plan, it is possible for the activity support device 20 to dynamically respond and propose the user a sufficient activity to achieve expenditure of the target physical activity amount each time as the exercise menu.

### [Third Modification]

By operating the user terminal 1a, the user may input a physical condition and mood of the day from the predetermined website or the predetermined application at any timing, and may transmit the input information to the activity support device 20. When the information concerning the physical condition or the like is obtained, the activity support device 20 adjusts the target physical activity amount or the intensity of a physical activity recommended, based on the information. Specifically, when the physical condition of the user is not feeling well, the activity support device 20 decreases the target physical activity amount or decreases the intensity of the recommended physical activity. As described above, by controlling the target physical activity amount and the intensity of the recommended physical activity, it is possible to make the physical activity a habit which is easy for the user.

In addition, by operating the activity support device 20, the user may input meal details from the predetermined website or the predetermined application at any timing, and transmits information concerning the meal details to the activity support device 20. Upon acquiring the information concerning the meal details, the activity support device 20 adjusts the target physical activity amount or the intensity of the recommended physical activity in consideration of calories intake based on the information. Specifically, the activity support device 20 increases the recommended intensity of the physical activity to increase a calorie expenditure when a calorie intake is high, compared to a case where the calorie intake is not high. According to this adjustment, the activity support device 20 can propose an appropriate physical activity to the user as the exercise menu in consideration of an energy balance when the user overeats or the like.

### [Fourth Modification]

In the first example embodiment described above, the activity determination unit 34 determines one physical activity recommended to the user from a plurality of physical activity options; however, the present disclosure is not limited thereto, and the activity support device 20 may determine two or more physical activities recommended to the user from the plurality of physical activity options. In this case, the activity support device 20 transmits information concerning a plurality of recommended physical activities to the user terminal 1a, and proposes the information to the user as exercise menu. According to this, it is possible for the user to select and perform the most preferable physical activity in the proposed exercise menu.

### [Fifth Modification]

Although not described in the first example embodiment described above, a machine learning model may be used to determine a physical activity recommended to the user. For instance, when the difference between the target physical activity amount and the total expended physical activity amount is input, the activity support device 20 may construct a recommended physical activity output model which outputs the optimized physical activity to compensate for the difference as the physical activity recommended to the user. Training data are used to construct (generate) the recommended physical activity output model. The training data are data which are associated with input data which are input for learning of the recommended physical activity output model and the correct answer data corresponding to the input data. In this variation, the input data represent the difference between the target physical activity amount and the total expended physical activity amount, and correct answer data represent a physical activity optimized to compensate for the difference between the target physical activity amount and the total physical activity amount. The activity support device 20 trains the recommended physical activity output model so as to output the optimized physical activity to compensate for the difference based on the difference between the target physical activity amount and the total expended physical activity amount, which is input as the input data. As a method of machine learning, for instance, a model using a neural network or the like is used. Accordingly, it is possible for the activity support device 20 to determine a physical activity recommended to the user based on the physical activity which is output by the recommended physical activity output model.

Note that the first modification to the fifth modification are not limited to the first example embodiment, these modifications can be applied to each of the second example embodiment and the third example embodiment.

A part or all of the example embodiments described above may also be described as the following supplementary notes, but not limited thereto.

### (Supplementary Note 1)

1. An activity support device comprising:
a plan acquisition means configured to acquire a target physical activity amount of a user in a predetermined period and a predictive physical activity amount representing an expected expenditure of a physical activity amount;
an expended physical activity amount acquisition means configured to acquire an expended physical activity amount of the user up to a current time in the predetermined period;
a total expended physical activity amount derivation means configured to derive a total expended physical activity amount of the user in the predetermined period, based on the predictive physical activity amount and the expended physical amount; and
an activity determination means configured to determine a physical activity to the user from among physical activity options by comparing the target physical amount and the total expended physical amount.

### (Supplementary Note 2)

2. The activity support device according to supplementary note 1, wherein the plan acquisition means acquires activity plan information concerning an activity plan of the user in the predetermined period, and derives the predictive physical activity amount based on a distance of a rout to a destination specified in the activity plan information from a current location of the user.

### (Supplementary Note 3)

3. The activity support device according to supplementary note 2, wherein the plan acquisition means acquires environmental information concerning a variation of a load on a road surface condition in the rout to the destination from the current location of the user, and derives the predictive physical activity amount in consideration of the variation of the load on the road surface condition.

### (Supplementary Note 4)

4. The activity support device according to supplementary note 1, wherein the processor executes further instructions to
request the user a report of a quality evaluation of a physical activity level with respect to the physical activity recommended to the user; and
derive a change proposal of the target physical activity amount in accordance with a result of the quality evaluation.

### (Supplementary Note 5)

5. The activity support device according to supplementary note 2, wherein the activity plan information includes a rout to the destination specified by the user.

### (Supplementary Note 6)

6. The activity support device according to supplementary note 2, further comprising an alert output means configured to output an alert for prompting to modify the activity plan in a case of location information of the current location of the user in the predetermined period that is different to location information of the user based on the activity plan information.

### (Supplementary Note 7)

7. The activity support device according to supplementary note 2, wherein in a case of location information of the current location of the user in the predetermined period that is different to location information of the user based on the activity plan information,
the plan acquisition means derives a new predictive physical activity amount based on the location information of the current location of the user,
the total expended physical activity amount derivation means derives a new total expended physical activity amount based on the new predictive physical activity amount and the expended physical activity amount, and
the activity determination means determines a physical activity recommended to the user from among the physical activity options by comparing the target physical activity amount and the new total expended physical activity amount.

### (Supplementary Note 8)

8. The activity support device according to supplementary note 1, wherein by using a machine learning model trained to output a physical activity optimized to compensate a difference in response to an input of the difference between the target physical activity amount and the total expended physical activity amount, the activity determination means determines a physical activity recommended to the user based on the activity output from the machine learning model.

### (Supplementary Note 9)

9. An activity support method comprising:
acquiring a target physical activity amount of a user in a predetermined period and a predictive physical activity amount representing an expected expenditure of a physical activity amount;
acquiring an expended physical activity amount of the user up to a current time in the predetermined period;
deriving a total expended physical activity amount of the user in the predetermined period, based on the predictive physical activity amount and the expended physical amount; and
determining a physical activity to the user from among physical activity options by comparing the target physical amount and the total expended physical amount.

### (Supplementary Note 10)

10. A non-transitory computer readable recording medium storing a program, the program causing a computer to perform a pipeline evaluation process comprising:
acquiring a target physical activity amount of a user in a predetermined period and a predictive physical activity amount representing an expected expenditure of a physical activity amount;
acquiring an expended physical activity amount of the user up to a current time in the predetermined period;
deriving a total expended physical activity amount of the user in the predetermined period, based on the predictive physical activity amount and the expended physical amount; and
determining a physical activity to the user from among physical activity options by comparing the target physical amount and the total expended physical amount.

While the present disclosure has been described with reference to the example embodiments and examples, the present disclosure is not limited to the above example embodiments and examples. Various changes which can be understood by those skilled in the art within the scope of the present disclosure can be made in the configuration and details of the present disclosure.

This application is based upon and claims the benefit of priority from Japanese Patent Application 2023-164177, filed on Sep. 27, 2023, the disclosure of which is incorporated herein in its entirety by reference.

### DESCRIPTION OF SYMBOLS

20, 20x, 20y Activity support device
5 Network
11, 21 Interface
12, 22 Processor
13, 23 Memory
14, 24 Recording medium
15, 25 Display unit
16, 26 Input unit
27 User DB

1. An activity support device comprising:
at least one memory configured to store instructions; and
at least one processor configured to execute the instructions to:
acquire a target physical activity amount of a user in a predetermined period and a predictive physical activity amount representing an expected expenditure of a physical activity amount;
acquire an expended physical activity amount of the user up to a current time in the predetermined period;
derive a total expended physical activity amount of the user in the predetermined period, based on the predictive physical activity amount and the expended physical amount; and
determine a physical activity to the user from among physical activity options by comparing the target physical amount and the total expended physical amount.

2. The activity support device according to claim 1, wherein the processor acquires activity plan information concerning an activity plan of the user in the predetermined period, and derives the predictive physical activity amount based on a distance of a rout to a destination specified in the activity plan information from a current location of the user.

3. The activity support device according to claim 2, wherein the processor acquires environmental information concerning a variation of a load on a road surface condition in the rout to the destination from the current location of the user, and derives the predictive physical activity amount in consideration of the variation of the load on the road surface condition.

4. The activity support device according to claim 1, wherein the processor executes further instructions to
request the user a report of a quality evaluation of a physical activity level with respect to the physical activity recommended to the user; and
derive a change proposal of the target physical activity amount in accordance with a result of the quality evaluation.

5. The activity support device according to claim 2, wherein the activity plan information includes a rout to the destination specified by the user.

6. The activity support device according to claim 2, wherein the processor further executes an instruction to output an alert for prompting to modify the activity plan in a case of location information of the current location of the user in the predetermined period that is different to location information of the user based on the activity plan information.

7. The activity support device according to claim 2, wherein with respect to location information of the current location of the user in the predetermined period that is different to location information of the user based on the activity plan information,
the processor derives a new predictive physical activity amount based on the location information of the current location of the user,
the processor derives a new total expended physical activity amount based on the new predictive physical activity amount and the expended physical activity amount, and
the processor determines a physical activity recommended to the user from among the physical activity options by comparing the target physical activity amount and the new total expended physical activity amount.

8. The activity support device according to claim 1, wherein by using a machine learning model trained to output a physical activity optimized to compensate a difference in response to an input of the difference between the target physical activity amount and the total expended physical activity amount, the processor determines a physical activity recommended to the user based on the activity output from the machine learning model.

9. An activity support method comprising:
acquiring a target physical activity amount of a user in a predetermined period and a predictive physical activity amount representing an expected expenditure of a physical activity amount;
acquiring an expended physical activity amount of the user up to a current time in the predetermined period;
deriving a total expended physical activity amount of the user in the predetermined period, based on the predictive physical activity amount and the expended physical amount; and
determining a physical activity to the user from among physical activity options by comparing the target physical amount and the total expended physical amount.

10. A non-transitory computer readable recording medium storing a program, the program causing a computer to perform a process comprising:
acquiring a target physical activity amount of a user in a predetermined period and a predictive physical activity amount representing an expected expenditure of a physical activity amount;
acquiring an expended physical activity amount of the user up to a current time in the predetermined period;
deriving a total expended physical activity amount of the user in the predetermined period, based on the predictive physical activity amount and the expended physical amount; and
determining a physical activity to the user from among physical activity options by comparing the target physical amount and the total expended physical amount.
